(19) United States
(12) Patent Application Publication
(10) Pub. No.: US 2003/0181501 A1
(43) Pub. Date: Sep. 25, 2003

(54) Intraorally disintegrating valdecoxib compositions

(76) Inventors: Trang T. Le, Vernon Hills, IL (US); Blake C. Ludwig, Gurnee, IL (US); Joseph P. Reo, Kalamazoo, MI (US); Uday J. Shah, Hoffman Estates, IL (US); Ken Yamamoto, Portage, MI (US)

Correspondence Address: PHARMACIA CORPORATION, GLOBAL PATENT DEPARTMENT, POST OFFICE BOX 1027, ST. LOUIS, MO, 63006, US

(21) Appl. No.: 10/252,222

(22) Filed: Sep. 23, 2002

Related U.S. Application Data
(60) Provisional application No. 60/325,356, filed on Sep. 26, 2001.

(51) Int. Cl. A61K 31/415; A61K 9/20

(52) U.S. Cl. 514/406; 424/465

(57) ABSTRACT

Orally disintegrating valdecoxib fast-melt tablets and processes for preparing such dosage forms are provided. The compositions are useful in treatment or prophylaxis of cyclooxygenase-2 mediated conditions and disorders

This application claims priority of U.S. provisional application serial No. 60/325,356 filed on Sep. 26, 2001.

### FIELD OF THE INVENTION

The present invention relates to intraorally disintegrating pharmaceutical compositions containing valdecoxib as an active ingredient, to processes for preparing such compositions, and to methods of treatment of cyclooxygenase-2 mediated disorders comprising orally administering such compositions to a subject.

### BACKGROUND OF THE INVENTION

The compound 4-(5-methyl-3-phenyl-4-isoxazolyl)benzenesulfonamide, also referred to herein as valdecoxib, was disclosed in U.S. Pat. No. 5,633,272 to Talley, et al., herein incorporated by reference, together with processes for preparing this and related compounds. Valdecoxib has the structure:

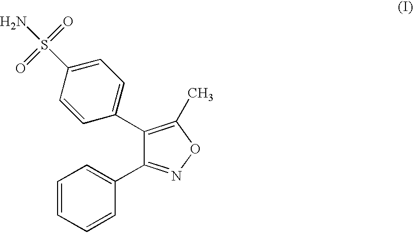

The compounds reported in above-cited U.S. Pat. No. 5,633,272, including valdecoxib, are disclosed therein as useful anti-inflammatory, analgesic and antipyretic drugs having a high degree of selectivity for inhibition of cyclooxygenase-2 (COX-2) over cyclooxygenase-1 (COX-1). Above-cited U.S. Pat. No. 5,633,272 also contains general references to formulations for the administration of such compounds, including orally deliverable dosage forms such as tablets and capsules.

Valdecoxib has extremely low solubility in water. See for example Dionne (1999), “COX-2 inhibitors—IBC Conference, Apr. 12-13, 1999, Coronado, Calif., U.S.A.”, IDrugs, 2(7), 664-666.

U.S. Pat. No. 5,576,014, incorporated herein by reference, discloses an intrabuccally dissolving compressed molding prepared by a wet granulation process wherein a low moldability saccharide is granulated with a high moldability saccharide to form a granulate, which is then compressed into a molding. The resulting molding can incorporate a drug and is said to show quick disintegration and dissolution in the buccal cavity but to maintain sufficient hardness so as not break during production and distribution. The compressed molding of U.S. Pat. No. 5,576,014 is a type of dosage form known as a “fast-melt tablet”, exhibiting rapid disintegration, usually associated with the carrier materials, typically sugars, and concomitant rapid dissolution or dispersion of the drug in the mouth, usually without need for water other than that contained in saliva. A drug formulated in such a tablet is readily swallowed.

Co-assigned International Patent Publication No. WO 01/41761 discloses orally deliverable valdecoxib compositions having fast-onset properties. None of the compositions disclosed therein is an intraorally disintegrating composition.

A well-known problem with many intraorally disintegrating compositions, even those containing sugars and/or sweetening and/or flavoring agents, is an unpleasant taste resulting from the presence of an active drug therein. Generally, as the amount of active drug present in a particular intraorally disintegrating dosage form decreases, and/or as the aqueous solubility of a drug decreases, the less bitter and/or sour will be the taste of the dosage form . See for example Lieberman et al. (1989), Pharmaceutical Dosage Forms: Tablets Vol. 1, pp. 381. Marcel Dekker, New York. Valdecoxib, a drug with very low water solubility and with relatively low dose requirements, would therefore be expected when formulated as an intraorally disintegrating composition to have acceptable or, at worst, only moderately unpleasant organoleptic properties. Surprisingly, however, we have now discovered that valdecoxib has an extremely unpleasant taste. Thus, there remains a need for intraorally disintegrating valdecoxib compositions having acceptable organoleptic properties.

Taste-masking technologies which act by inhibiting oral dissolution of moderately or highly water soluble drugs have been applied to pharmaceutical dosage forms. See for example Lieberman et al. (1989), op. cit. In such cases, improved taste is believed to result from a decrease in the amount of drug which dissolves in the mouth prior to entry into the gastrointestinal tract. Given the already extremely low aqueous solubility of valdecoxib, however, it was not expected that any further reduction in oral dissolution of valdecoxib would lead to improved organoleptic properties. Further, it was expected that additional reduction in aqueous solubility of valdecoxib would result in unacceptable delay of therapeutic onset. Surprisingly, however, we have now discovered processes for preparing organoleptically acceptable intraorally disintegrating valdecoxib compositions, which compositions exhibit improved organoleptic properties, yet which still exhibit rapid onset of therapeutic effect.

### SUMMARY OF THE INVENTION

Accordingly, there is now provided a process for preparing an intraorally disintegrating valdecoxib composition (e.g. a fast-melt tablet), the process comprising a step of providing valdecoxib in particulate form; a step of adding to the valdecoxib a pharmaceutically acceptable dissolution retardant to form a valdecoxib composite; a step of admixing with the valdecoxib composite at least one pharmaceutically acceptable excipient that exhibits rapid oral dissolution, said admixing step forming a tableting blend; a step of granulating the valdecoxib, valdecoxib composite, or tableting blend; and a step of compressing the tableting blend to form a tablet. In the process of the invention, the granulating step occurs prior to, simultaneously with, and/or after said step of adding the dissolution retardant. Compositions prepared by such a process represent an embodiment of the present invention.

In a preferred embodiment, the granulation step comprises wet granulation and the process further comprises a step of drying the valdecoxib composite or tableting blend during and/or after the wet granulation step.

There is also now provided an intraorally disintegrating composition comprising (a) particulate valdecoxib in a therapeutically effective amount, (b) at least one pharmaceutically acceptable dissolution retardant, and (c) at least one pharmaceutically acceptable excipient which exhibits rapid oral dissolution; wherein the composition is organoleptically acceptable. The composition is preferably a fast-melt tablet.

A particularly useful intraorally disintegrating composition of the present invention is a rapidly disintegrating oral dosage form that dissolves in the mouth without need for drinking water or other fluid (e.g. a fast-melt). The term “fast-melt” as used herein refers to a composition such as a tablet wherein an active agent or drug is distributed or dispersed in a matrix formed by a carrier that, upon oral administration of the composition to a subject, disintegrates in the oral cavity, thereby releasing the drug, typically in particulate form, for entry to the gastrointestinal tract by swallowing, and subsequent absorption. The term “oral cavity” includes the entire interior of the mouth, including not only the buccal cavity (that part of the oral cavity anterior to the teeth and gums) but also the sublingual and supralingual spaces.

An “organoleptically acceptable” dosage form or a dosage form having “acceptable organoleptic properties” herein is one that, upon intraoral interaction in an amount providing a single dose of the therapeutic agent, does not have an excessively unpleasant taste, smell or mouth feel, for example a pronouncedly bitter taste, as perceived by a majority of human subjects, or as determined by analysis of a blind taste evaluation study as is described hereinbelow.

Processes and compositions of the invention have been found to overcome the unacceptable organoleptic properties of valdecoxib without unacceptably sacrificing rapid onset characteristics or therapeutic effectiveness. Thus, in a significant advance in the art, valdecoxib is now presented in an organoleptically acceptable fast-melt formulation. Particular advantages of compositions of the invention is that they have improved organoleptic properties yet do not exhibit substantially increased time to therapeutic onset, and such compositions can be efficiently prepared by processes described herein.

### DETAILED DESCRIPTION OF THE INVENTION

As indicated above, the present invention provides a process for preparing an intraorally disintegrating valdecoxib dosage form, preferably a fast-melt tablet. The process comprises a step of providing valdecoxib in particulate form; a step of adding to the valdecoxib a pharmaceutically acceptable dissolution retardant to form a valdecoxib composite; a step of admixing with the valdecoxib composite at least one pharmaceutically acceptable excipient that exhibits rapid oral dissolution, said admixing step forming a tableting a blend; a step of granulating the valdecoxib, valdecoxib composite, or tableting blend; and a step of compressing the tableting blend to form a tablet. The granulating step occurs prior to, simultaneously with, and/or after said step of adding the dissolution retardant.

A further embodiment of the invention is an oral fast-melt composition comprising (a) particulate valdecoxib in a therapeutically effective amount, (b) at least one pharmaceutically acceptable dissolution retardant, and (c) at least one pharmaceutically acceptable excipient which exhibits rapid oral dissolution; wherein the composition is organoleptically acceptable. Preferably, the at least one pharmaceutically acceptable dissolution retardant is in intimate association with the valdecoxib in the composition.

An “intimate association” in the present context includes, for example, valdecoxib admixed with the dissolution retardant, valdecoxib embedded or incorporated in the dissolution retardant, valdecoxib forming a coating on particles of the dissolution retardant or vice versa, and a substantially homogeneous dispersion of valdecoxib throughout the dissolution retardant. Valdeocoxib in intimate association with a dissolution retardant is also referred to herein as a “valdecoxib composite”. The term “substantially homogeneous” herein with reference to a composite or pharmaceutical composition that comprises multiple components means that the components are sufficiently mixed such that individual components are not present as discrete layers and do not form concentration gradients within the composition.

Another related embodiment of the invention provides an intraorally disintegrating composition comprising (a) particulate valdecoxib in a therapeutically effective amount, (b) at least one pharmaceutically acceptable dissolution retardant, and (c) at least one pharmaceutically acceptable excipient which exhibits rapid oral dissolution; wherein the composition is organoleptically acceptable; and wherein the composition disintegrates within about 60 seconds, preferably within about 30 seconds, and more preferably within about 15 seconds, after placement in the oral cavity of a human subject.

Another related embodiment of the invention provides an intraorally disintegrating composition comprising (a) particulate valdecoxib in a therapeutically effective amount, (b) at least one pharmaceutically acceptable dissolution retardant, and (c) at least one pharmaceutically acceptable excipient which exhibits rapid oral dissolution; wherein the composition is organoleptically acceptable; and wherein the composition, when placed in United States Pharmacopeia 24 in vitro disintegration Test Number 701, exhibits a disintegration time of less than about 300 seconds, preferably less than about 200 seconds, and more preferably less than about 100 seconds.

Another embodiment of the invention provides an intraorally disintegrating composition comprising (a) particulate valdecoxib in a therapeutically effective amount, (b) at least one pharmaceutically acceptable dissolution retardant, and (c) at least one pharmaceutically acceptable excipient which exhibits rapid oral dissolution; wherein the composition is organoleptically acceptable; and wherein administration of the composition to a human subject results in a valdecoxib threshold concentration for therapeutic effect within about 0.5 h, preferably within about 0.3 h, of administration.

By “a threshold concentration for therapeutic effect” is meant a minimum concentration of valdecoxib in blood serum consistent with therapeutic benefit for the particular indication for which the valdecoxib is administered. Typically this threshold concentration is at least about 20 ng/ml, for example about 25 ng/ml to about 75 ng/ml.

It will be understood that the amount of valdecoxib in a dose unit effective to provide a threshold concentration for therapeutic effect is dependent, inter alia, on the body weight of the treated subject. Where the subject is a child or a small animal (e.g., a dog), for example, an amount of valdecoxib relatively low in the therapeutically effective range of about 1 mg to about 100 mg is likely to provide blood serum concentrations consistent with threshold concentration and Cₘₐₓ criteria. Where the subject is an adult human or a large animal (e.g., a horse), the indicated blood serum concentrations of valdecoxib are likely to require a relatively greater dosage amount of valdecoxib. For an adult human, a suitable amount of valdecoxib per dose in a composition of the present invention to provide the indicated blood serum concentrations is typically about 5 mg to about 40 mg.

A related embodiment of the invention provides an intraorally disintegrating composition comprising (a) particulate valdecoxib in a therapeutically effective amount, (b) at least one pharmaceutically acceptable dissolution retardant, and (c) at least one pharmaceutically acceptable excipient which exhibits rapid oral dissolution; wherein the composition is organoleptically acceptable; and wherein administration of the composition to a human subject results in a maximum blood serum concentration (Cₘₐₓ) not less than about 100 ng/ml, preferably not less than about 200 ng/ml, and more preferably not less than about 300 ng/ml.

Another related embodiment of the invention provides an intraorally disintegrating composition comprising (a) particulate valdecoxib in a therapeutically effective amount, (b) at least one pharmaceutically acceptable dissolution retardant, and (c) at least one pharmaceutically acceptable excipient which exhibits rapid oral dissolution; wherein the composition is organoleptically acceptable; and wherein administration of the composition to a human subject results in a time to reach maximum blood serum concentration (Tₘₐₓ) not greater than about 5 h, preferably not greater than about 4.5 h, more preferably not greater than about 4 h, and still more preferably not greater than about 3 h.

Ingredients of Compositions of the Invention

A composition of the invention comprises valdecoxib as active ingredient, at least one pharmaceutically acceptable dissolution retardant, and at least one pharmaceutically acceptable excipient which exhibits rapid oral dissolution. Optionally, a composition of the invention can contain one or more additional pharmaceutically acceptable excipients including, but not limited to, water-soluble lubricants, water-insoluble lubricants, disintegrants, glidants, sweeteners, flavoring agents, colorants, etc. Such optional additional components should be physically and chemically compatible with the other ingredients of the composition and must not be deleterious to the recipient.

Valdecoxib

Processes and compositions of the invention are particularly suitable for valdecoxib as the active drug. Processes for preparing particulate valdecoxib are known per se, for example as is described in above-cited U.S. Pat. No. 5,474,995, incorporated herein by reference. Importantly, any solid state form of valdecoxib, illustratively can be used in processes and compositions of the invention, illustratively any form described in International Patent Publication No. 98/06708, incorporated herein by reference.

A valdecoxib dosage unit of the invention comprises valdecoxib in a therapeutically effective amount of about 1 mg to about 100 mg, preferably about 5 mg to about 50 mg. Compositions of the invention contain valdecoxib in particulate form. Primary valdecoxib particles, generated for example by milling or grinding, or by precipitation from solution, can agglomerate to form secondary aggregate particles. The term “particle size” as used herein refers to size, in the longest dimension, of primary particles, unless the context demands otherwise. Particle size is believed to be an important parameter affecting clinical effectiveness of valdecoxib. Thus, in one embodiment, a valdecoxib dosage form has a distribution of valdecoxib particle sizes such that the D₉₀ particle size is not greater than about 75 μm. The “D₉₀ particle size” is defined herein as a particle size such that 90% by weight of the particles are smaller, in their longest dimension, than that particle size.

In addition or alternatively, valdecoxib particles in a dosage form of the to invention preferably have a weight average particle size of about 1 μm to about 10 μm, most preferably about 5 μm to about 7 μm.

Dissolution Retardant

Any pharmaceutically acceptable excipient which, when in intimate association with valdecoxib, retards, inhibits or slows dissolution of valdecoxib in water, can be used as a dissolution retardant in processes and compositions of the invention. Preferably, the dissolution retardant is a polymer. Non-limiting illustrative examples of suitable polymers for use as dissolution retardants include polymethacrylates, for example Eudragit® E PO of Röhm, ethylcellulose, for example Surelease® of Colorcon, hydroxypropylmethylcellulose (HPMC), polyvinylpyrrolidone (PVP), hydroxypropylethylcellulose, and hydroxypropylcellulose. Eudragit® E PO (ammonio methacrylate copolymer or methacrylic acid copolymer) or an equivalent polymethacrylate product is a particularly preferred dissolution retardant.

The at least one dissolution retardant is typically present in a total amount of about 0.5% to about 15%, preferably about 0.75% to about 10%, and more preferably about 1.0% to about 5%, by weight of the composition.

Excipients Which Exhibit Rapid Oral Dissolution

Suitable excipients which exhibit rapid oral dissolution are those pharmaceutically acceptable excipients which are soluble, freely soluble, or very soluble in water, for example as described in Ansel et al. (1995) Pharmaceutical Dosage Forms and Drug Delivery Systems 6th Ed, pp. 228. Williams & Wilkins, Baltimore. Preferably, such excipients have a sweet taste. A presently preferred class of excipients which exhibit rapid oral dissolution for use in compositions and processes of the invention are carbohydrates. Particularly preferred excipients which exhibit rapid oral dissolution are saccharides including both low moldability and high moldability saccharides.

Presently preferred low moldability saccharides include lactose and mannitol, particularly mannitol in its non-direct compression or powder form as described in Kibbe (2000) Handbook of Pharmaceutical Excipients, 3rd Ed., Pharmaceutical Press, pp. 324-328. Presently preferred high moldability saccharides include maltose, maltitol and sorbitol. Alternatively, certain oligosaccharides can be useful. The oligosaccharide used is not particularly limited so long as it shows rapid dissolution in the oral cavity and consists of two or more monosaccharide residues. Where an oligosaccharide is used, one consisting of 2 to 6 monosaccharide residues is preferable, and the type and combination of monosaccharide residues constituting the oligosaccharide are not limited. Particularly preferred high moldability saccharides are maltose and maltitol, more particularly maltose.

Where both a high moldability saccharide and low moldability saccharide are present in a composition of the invention, the weight ratio of high moldability saccharide to low moldability saccharide is important in maintaining a combination of acceptable tablet hardness and rapid intraoral disintegration. A suitable ratio is about 2 to about 20 parts by weight, preferably about 5 to about 10 parts by weight, and more preferably about 5 to about 7.5 parts by weight, of the high moldability saccharide per 100 parts by weight of the low moldability saccharide.

If the ratio of high to low moldability saccharide is less than about 2:100 by weight, tablets typically do not achieve their desired hardness, resulting in increased breakage during storage, transportation or handling. Alternatively, if the ratio of high to low moldability saccharide exceeds about 20:100 by weight, the tablets become too hard and desired rapid disintegration in the oral cavity is not achieved.

One or more excipients which exhibit rapid oral dissolution are typically present in compositions of the invention in a total amount of about 10% to about 90%, preferably about 10% to about 80%, and more preferably about 10% to about 75%.

Wetting Agents

Compositions of the present invention optionally comprise one or more pharmaceutically acceptable wetting agents. Surfactants, hydrophilic polymers and certain clays can be useful as wetting agents to aid in wetting of a hydrophobic drug, such as valdecoxib, by the granulation fluid during wet granulation. Where compositions of the present invention are made by the fluid bed granulation process, it is particularly advantageous that the composition contain a wetting agent.

Non-limiting examples of surfactants that can be used as wetting agents in compositions of the present invention include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and octoxynol 9, poloxamers (polyoxyethylene and polyoxypropylene block copolymers), polyoxyethylene fatty acid glycerides and oils, for example polyoxyethylene (8) caprylic/capric mono- and diglycerides (e.g., Labrasol™ of Gattefossé), polyoxyethylene (35) castor oil and polyoxyethylene (40) hydrogenated castor oil; polyoxyethylene alkyl ethers, for example polyoxyethylene (20) cetostearyl ether, polyoxyethylene fatty acid esters, for example polyoxyethylene (40) stearate, polyoxyethylene sorbitan esters, for example polysorbate 20 and polysorbate 80 (e.g., Tween™ 80 of ICI), propylene glycol fatty acid esters, for example propylene glycol laurate (e.g., Lauroglycol™ of Gattefosse), sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. Sodium lauryl sulfate is a preferred wetting agent in compositions of the present invention.

One or more wetting agents, if desired, are typically present in compositions of the present invention in a total amount of about 0.05% to about 5%, preferably about 0.075% to about 2.5%, and more preferably about 0.25% to about 1%, for example about 0.5%, by weight of the composition.

Water-insoluble Lubricants

Compositions of the present invention optionally comprise one or more pharmaceutically acceptable water-insoluble lubricants as a carrier material. Suitable water-insoluble lubricants include, either individually or in combination, glyceryl behapate (e.g. Compritol™ 888), stearates (magnesium, calcium, and sodium), stearic acid, hydrogenated vegetable oils (e.g., Sterotex™), colloidal silica, talc, waxes and mixtures thereof. Optionally a water-insoluble lubricant can be used in mixture with a wetting agent, as for example in calcium stearate/sodium lauryl sulfate mixtures (e.g., Sterowet™).

Magnesium stearate, stearic acid and mixtures thereof are preferred water-insoluble lubricants.

One or more water-insoluble lubricants optionally are present in compositions of the present invention in a typical total amount of about 0.05% to about 5%, preferably about 0.75% to about 2.5%, and more preferably about 1% to about 2%, for example, about 1.5%, by weight of the composition.

Water-soluble Lubricants

Compositions of the present invention optionally comprise one or more pharmaceutically acceptable water-soluble lubricants. Water-soluble lubricants can help to improve tablet dissolution characteristics. Water-soluble lubricants that can be used in compositions of the present invention either individually or in combination include, for example, boric acid, sodium benzoate, sodium acetate, sodium fumarate, sodium chloride, DL-leucine, polyethylene glycols (e.g., Carbowax™ 4000 and Carbowax™ 6000), and sodium oleate.

Disintegrants

Compositions of the present invention optionally comprise one or more pharmaceutically acceptable disintegrants. However, the oral fast-melt tablets provided herein typically disintegrate rapidly in the oral cavity and have no requirement for added disintegrant. Suitable disintegrants, if desired, include, either individually or in combination, starches, sodium starch glycolate, clays (such as Veegum™ HV), celluloses (such as purified cellulose, methylcellulose, sodium carboxymethylcellulose and carboxymethylcellulose), croscarmellose sodium, alginates, pregelatinized corn starches (such as National™ 1551 and National™ 1550), crospovidone, and gums (such as agar, guar, locust bean, karaya, pectin and tragacanth gums). Disintegrants can be added at any suitable step during the preparation of the composition, particularly prior to granulation or during a blending step prior to tablet compression. Croscarmellose sodium and sodium starch glycolate are preferred disintegrants.

One or more disintegrants optionally are present in a total amount of about 0.05% to about 15%, preferably about 0.5% to about 10%, and more preferably about 1% to about 3.5%, by weight of the composition.

Glidants

Compositions of the present invention optionally comprise one or more pharmaceutically acceptable glidants, for example to enhance flow of tableting material into tablet dies, to prevent sticking of tableting material to punches and dies, or to produce tablets having a sheen. Glidants may be added at any suitable step during preparation of the composition, particularly prior to granulation or during a blending step prior to tablet compression.

Without being bound by theory, it is believed that, in some situations, glidants, for example talc or silicon dioxide, act to reduce interfacial tension between drug particles, having the effect of inhibiting and/or reducing drug agglomeration, act to decrease electrostatic charges on the surface of drug powders, and act to reduce interparticular friction and surface rugosity of drug particles. See, for example, York (1975) J. Pharm. Sci., 64(7), 1216-1221.

Silicon dioxide is a preferred glidant. Suitable silicon dioxide products for use in preparing compositions of the invention include fumed silica or colloidal silica (e.g., Cab-O-Sil™ of Cabot Corp. and Aerosil™ of Degussa). Silicon dioxide, when present in compositions of the invention, is present in a total amount of about 0.05% to about 5%, preferably about 0.1% to about 2%, and more preferably about 0.25% to about 1%, for example, about 0.5%, by weight of the composition.

Sweetening Agents

Compositions of the present invention optionally comprise one or more pharmaceutically acceptable sweeteners. Non-limiting examples of sweeteners that can be used in compositions of the present invention include mannitol, propylene glycol, sodium saccharin, acesulfame K, neotame, aspartame, etc.

Flavoring Agents

Compositions of the present invention optionally comprise one or more pharmaceutically acceptable flavoring agents. Non-limiting examples of flavoring agents that can be used in compositions of the present invention include peppermint, spearmint, grape, cherry, strawberry, lemon, etc.

Tablet Characteristics

Size and Shape

In a preferred embodiment, compositions of the invention are in the form of discrete solid dosage units, most preferably tablets. Tablets of the invention can be made to any desired size, for example 8 mm, 10 mm, 12 mm, etc.; shape, for example round, oval, oblong, etc.; weight; and thickness. Optionally, solid dosage units of the invention may have etchings or monograms on one or both sides.

Disintegration

Preferred tablet compositions of the invention disintegrate in less than 300 seconds, preferably less than about 200 seconds, and more preferably less than about 100 seconds, for example about 30 seconds after placement in a standard in vitro disintegration assay (e.g., conducted according to U.S. Pharmacopeia 24 (2000), Test No. 701).

Alternatively or additionally, preferred fast-melt compositions of the invention disintegrate within about 60 seconds, preferably within about 30 seconds, and more preferably within about 15 seconds after placement in the oral cavity of a subject.

Hardness

Solid dosage forms of the invention have a hardness that can depend on size and shape as well as on composition, among other characteristics. Tablet hardness can be measured by any method known in the art, for example by a tablet hardness meter (e.g., Schleuniger). Preferably, compositions of the invention have a hardness of about 1 to about 10 kp, and more preferably of about 1 to about 6 kp.

In a presently preferred embodiment, solid dosage forms of the invention have sufficient hardness for handling and, therefore, can be put into practical use in the same manner as the case of ordinary tablets. The term “sufficient hardness for handling” as used herein means a hardness which can withstand removal from at least a standard type of blister packaging, or such a hardness as will withstand other handling such as packaging, delivery, carrying and the like.

Tablets of the invention preferably have a minimum hardness so as to resist breakage of the tablet during removal from standard blister packaging by pushing the tablet through a cover sheet. A suitable hardness is about 1 kp or more for a tablet having a diameter of about 8 mm, about 1.5 kp or more for a tablet having a diameter of about 10 mm, and about 2 kp or more when the tablet has a diameter of about 12 mm.

In another presently preferred embodiment, tablets of the invention have sufficient hardness such that a plurality of such tablets can be packaged together, for example in a glass or plastic bottle, without individual packaging, yet do not exhibit substantial breakage or sticking and/or melding together during normal shipping and handling. Tablets intended for such packaging preferably have a hardness of about 3 kp or more.

Packaging

Compositions of the invention can be packaged in any suitable manner known in the art. For example, a multiplicity of fast-melt tablets can be packaged together, for example in a glass or plastic bottle or container. Alternatively, fast-melt tablets of the invention can be individually wrapped, for example in plastic or foil, or packaged in known forms of blister packaging. Blister packaging with improved force distribution properties such as is disclosed in U.S. Pat. No. 5,954,204 to Grabowski, incorporated herein by reference, can be especially useful to package fast-melt tablets of the invention.

Administration of Fast-melt Tablets

Compositions of the present invention can be taken by a subject by any oral administration means in accordance with the subject's choice or condition. For example, fast-melt tablets of the invention can be taken without water. Upon placement in the oral cavity and especially in the cheek or above the tongue, such a tablet is exposed to saliva and rapidly disintegrates and dissolves therein. The rate of disintegration and/or dissolution increases further when an intraoral pressure, for example a pressure between the palate and tongue or a licking or sucking pressure, is applied to the tablet.

Alternatively, a tablet of the present invention can be taken with the aid of water in an amount sufficient to wet the oral cavity and to assist in disintegration of the tablet. Also, a tablet of the invention can be swallowed together with a small amount of water after complete or partial disintegration in the oral cavity. Compositions of the invention can also be swallowed directly with water.

Method to Make Fast-melt Tablets

The process described below is a non-limiting, illustrative method to make valdecoxib fast-melt tablets of the invention. Importantly, specific settings and parameters of the production process can be readily optimized by one of skill in the art in order to produce tablets with particularly desired characteristics.

In this illustrative process, valdecoxib and microcrystalline cellulose are de-lumped in a mill or grinder and blended to form a drug powder mixture. Next, the drug powder mixture is granulated, illustratively by roller compaction, slugging, high shear wet granulation, or fluid bed granulation. Where wet granulation is used, the drug powder mixture can be granulated with a solution or solution/suspension comprising a dissolution retardant and a wetting agent, for example sodium lauryl sulfate, to form granules. If the granules are not dried during granulation, for example as is the case in fluid bed granulation, they are dried after granulation, for example in an oven. The resulting dried granules are then milled to form a milled granulate. The milled granulate is then optionally blended with excipients which exhibit rapid oral dissolution, for example granulated mannitol and/or maltose, flavor, sweetener and lubricants in a tumble blender to form a tableting blend. The resulting tableting blend is then compressed on a rotary tablet press to a target tablet weight and hardness. The resulting tablets are then subjected to treatment, for example air flow treatment, in a humidity-controlled chamber with the effect of increasing tablet hardness.

Wet Granulation

Fluid bed granulation and high shear granulation are preferred methods of wet granulation in processes of the invention, although any known wet granulation method, for example pan granulation, can be used.

Illustratively, in fluid bed granulation, valdecoxib, silicon dioxide, and any other desired excipients are mixed together and sized in a mill or grinder. Next, the resulting drug powder mixture is granulated in a fluid bed by spraying a liquid solution or solution/suspension comprising a dissolution retardant and a wetting agent onto the mixture. The wet granules are then fluid bed dried. Importantly, the excipient exhibiting rapid oral dissolution, for example mannitol and/or maltose, can be dissolved in the liquid solution, or can be dry blended with the dry granules prior to compression.

After fluid bed granulation is complete, the resulting dried granules are then blended with any further desired excipients and then compressed into tablets.

Alternatively, in high-shear wet granulation, valdecoxib and any other desired excipients are blended under high shear in a granulator. Next, a liquid solution of dissolution retardant and wetting agent are added to the resulting drug powder mixture under continuing high shear, thereby forming wet granules.

After high-shear granulation is complete, the resulting granules are then dried, for example, in an oven, microwave or fluid bed. The dried granules are then transferred to a blender for addition of any other desired excipients to form a tableting blend, which is then compressed.

Whether fluid bed or high-shear granulation is used, the valdecoxib and excipient(s) exhibiting rapid dissolution can, in an alternative process, be separately granulated and the resulting granules mixed together prior to compression.

Tablet Compression

Compression is the process by which an appropriate volume of a tableting blend produced as described above is compressed between an upper and lower punch to consolidate material into a single solid dosage form such as a tablet. In processes for manufacture of fast-melt tablets of the present invention, any suitable means for compression can be used including, for example, a single punch tablet machine or a high speed rotary tablet press. The tableting pressure is not limited, and an appropriate pressure can be selected depending on the desired hardness and dissolution properties of the resulting tablets. Where tablets are to undergo temperature and humidity treatment as described immediately below, the tablets are preferably compressed to an initial hardness (prior to temperature and humidity treatment) of about 0.75 to about 1.5 kp.

Temperature and Humidity Treatment

Optionally, tablets of the invention can undergo heat and humidity treatment after the tablet compression step. Such treatment can be performed in a humidity chamber, for example, to increase hardness of the tablets. Illustratively, during this treatment, tablets are first subjected to low temperature, high humidity air flow conditions, for example, about 25° C. to about 32° C. and about 80% relative humidity, for a period of about 45 to about 120 minutes. Tablets are then subjected to high temperature, low humidity conditions, for example about 35° C. to about 50° C. and 30% relative humidity for a period of about 45 to about 120 minutes. Without being bound by theory, it is believed that treatment of fast-melt tablets in a low temperature/high humidity chamber followed by treatment in a high temperature/low humidity chamber increases tablet hardness and reduces tablet friability without sacrificing desired fast-melt characteristics such as rapid disintegration and rapid dissolution.

Utility of Compositions of the Invention

Fast-melt tablets, herein also referred to as compositions, of the present invention are useful in treatment and prevention of a very wide range of disorders mediated by cyclooxygenase-2 (COX-2), including but not restricted to disorders characterized by inflammation, pain and/or fever. Such compositions are especially useful as anti-inflammatory agents, such as in treatment of arthritis, with the additional benefit of having significantly less harmful side effects than compositions of conventional nonsteroidal anti-inflammatory drugs (NSAIDs) that lack selectivity for COX-2 over COX-1. In particular, such compositions have reduced potential for gastrointestinal toxicity and gastrointestinal irritation including upper gastrointestinal ulceration and bleeding, reduced potential for renal side effects such as reduction in renal function leading to fluid retention and exacerbation of hypertension, reduced effect on bleeding times including inhibition of platelet function, and possibly a lessened ability to induce asthma attacks in aspirin-sensitive asthmatic subjects, by comparison with compositions of conventional NSAIDs. Thus compositions of the invention comprising a selective COX-2 inhibitory drug are particularly useful as an alternative to conventional NSAIDs where such NSAIDs are contraindicated, for example in patients with peptic ulcers, gastritis, regional enteritis, ulcerative colitis, diverticulitis or with a recurrent history of gastrointestinal lesions; gastrointestinal bleeding, coagulation disorders including anemia such as hypoprothrombinemia, hemophilia or other bleeding problems; kidney disease; or in patients prior to surgery or patients taking anticoagulants.

Such compositions are useful to treat arthritic disorders, including but not limited to rheumatoid arthritis, spondyloarthropathies, gouty arthritis, osteoarthritis, systemic lupus erythematosus and juvenile arthritis.

Such compositions are also useful in treatment of asthma, bronchitis, menstrual cramps, preterm labor, tendinitis, bursitis, allergic neuritis, cytomegalovirus infectivity, apoptosis including HIV-induced apoptosis, lumbago, liver disease including hepatitis, skin-related conditions such as psoriasis, eczema, acne, burns, dermatitis and ultraviolet radiation damage including sunburn, and post-operative inflammation including that following ophthalmic surgery such as cataract surgery or refractive surgery.

Such compositions are useful to treat gastrointestinal conditions such as inflammatory bowel disease, Crohn's disease, gastritis, irritable bowel syndrome and ulcerative colitis.

Such compositions are useful in treating inflammation in such diseases as migraine headaches, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephrotic syndrome, Behcet's syndrome, polymyositis, gingivitis, nephritis, hypersensitivity, swelling occurring after injury including brain edema, myocardial ischemia, and the like.

Such compositions are useful in treatment of ophthalmic diseases, such as retinitis, scleritis, episcleritis, conjunctivitis, retinopathies, uveitis, ocular photophobia, and of acute injury to eye tissue.

Such compositions are useful in treatment of pulmonary inflammation, such as that associated with viral infections and cystic fibrosis, and in bone resorption such as that associated with osteoporosis.

Such compositions are useful for treatment of certain central nervous system disorders, such as cortical dementias including Alzheimer's disease, neurodegeneration, and central nervous system damage resulting from stroke, ischemia and trauma. The term “treatment” in the present context includes partial or total inhibition of dementias, including Alzheimer's disease, vascular dementia, multi-infarct dementia, pre-senile dementia, alcoholic dementia and senile dementia.

Such compositions are useful in treatment of allergic rhinitis, respiratory distress syndrome, endotoxin shock syndrome and liver disease.

Such compositions are useful in treatment of pain, including but not limited to postoperative pain, dental pain, muscular pain, and pain resulting from cancer. For example, such compositions are useful for relief of pain, fever and inflammation in a variety of conditions including rheumatic fever, influenza and other viral infections including common cold, low back and neck pain, dysmenorrhea, headache, toothache, sprains and strains, myositis, neuralgia, synovitis, arthritis, including rheumatoid arthritis, degenerative joint diseases (osteoarthritis), gout and ankylosing spondylitis, bursitis, burns, and trauma following surgical and dental procedures.

Such compositions are useful for, but not limited to, treating and preventing inflammation-related cardiovascular disorders in a subject. Such compositions are useful for treatment and prevention of vascular diseases, coronary artery disease, aneurysm, vascular rejection, arteriosclerosis, atherosclerosis including cardiac transplant atherosclerosis, myocardial infarction, embolism, stroke, thrombosis including venous thrombosis, angina including unstable angina, coronary plaque inflammation, bacterial-induced inflammation including Chlamydia-induced inflammation, viral induced inflammation, and inflammation associated with surgical procedures such as vascular grafting including coronary artery bypass surgery, revascularization procedures including angioplasty, stent placement, endarterectomy, or other invasive procedures involving arteries, veins and capillaries.

Such compositions are useful for, but not limited to, treatment of angiogenesis-related disorders in a subject, for example to inhibit tumor angiogenesis. Such compositions are useful for treatment of neoplasia, including metastasis; ophthalmological conditions such as corneal graft rejection, ocular neovascularization, retinal neovascularization including neovascularization following injury or infection, diabetic retinopathy, macular degeneration, retrolental fibroplasia and glaucoma, including neovascular glaucoma; ulcerative diseases such as gastric ulcer; pathological, but non-malignant, conditions such as hemangiomas, including infantile hemangiomas, angiofibroma of the nasopharynx and avascular necrosis of bone; and disorders of the female reproductive system such as endometriosis.

Such compositions are useful for prevention or treatment of benign and malignant tumors/neoplasia including cancers, for example colorectal cancer, brain cancer, bone cancer, epithelial cell-derived neoplasia (epithelial carcinoma) such as basal cell carcinoma, adenocarcinoma, gastrointestinal cancer such as lip cancer, mouth cancer, esophageal cancer, small bowel cancer, stomach cancer, colon cancer, liver cancer, bladder cancer, pancreas cancer, ovary cancer, cervical cancer, lung cancer, breast cancer and skin cancer, such as squamous cell and basal cell cancers, prostate cancer, renal cell carcinoma, and other known cancers that affect epithelial cells throughout the body. Neoplasias for treatment of which compositions of the invention are contemplated to be particularly useful are gastrointestinal cancer, Barrett's esophagus, liver cancer, bladder cancer, pancreas cancer, ovary cancer, prostate cancer, cervical cancer, lung cancer, breast cancer and skin cancer, such as squamous cell and basal cell cancers. Compositions of the invention can also be used to treat fibrosis that occurs with radiation therapy. Such compositions can be used to treat subjects having adenomatous polyps, including those with familial adenomatous polyposis (FAP). Additionally, such compositions can be used to prevent polyps from forming in patients at risk of FAP.

Such compositions inhibit prostanoid-induced smooth muscle contraction by preventing synthesis of contractile prostanoids and hence can be of use in treatment of dysmenorrhea, premature labor, asthma and eosinophil-related disorders. They also can be of use for decreasing bone loss particularly in postmenopausal women (i.e., treatment of osteoporosis), and for treatment of glaucoma.

Preferred uses for compositions of the present invention are for treatment of rheumatoid arthritis and osteoarthritis, for pain management generally (particularly post-oral surgery pain, post-general surgery pain, post-orthopedic surgery pain, and acute flares of osteoarthritis), for treatment of Alzheimer's disease, and for colon cancer chemoprevention.

Besides being useful for human treatment, compositions of the invention are also useful for veterinary treatment of companion animals, exotic animals, farm animals, and the like, particularly mammals including rodents. More particularly, compositions of the invention are useful for veterinary treatment of cyclooxygenase-2 mediated disorders in horses, dogs and cats.

The present invention also is directed to a therapeutic method of treating a condition or disorder where treatment with a cyclooxygenase-2 inhibitory drug is indicated, the method comprising oral administration of one or more compositions of the present invention to a patient in need thereof. The dosage regimen to prevent, give relief from, or ameliorate the condition or disorder preferably corresponds to once-a-day or twice-a-day treatment, but can be modified in accordance with a variety of factors. These include the type, age, weight, sex, diet and medical condition of the patient and the nature and severity of the disorder. Thus, the dosage regimen actually employed can vary widely and can therefore deviate from the preferred dosage regimens set forth above.

Initial treatment of a patient suffering from a condition or disorder where treatment with a cyclooxygenase-2 inhibitory drug is indicated can begin with a dose regimen as indicated above. Treatment is generally continued as necessary over a period of several weeks to several months or years until the condition or disorder has been controlled or eliminated. Patients undergoing treatment with a composition of the invention can be routinely monitored by any of the methods well known in the art to determine the effectiveness of therapy. Continuous analysis of data from such monitoring permits modification of the treatment regimen during therapy so that optimally effective amounts of the drug are administered at any point in time, and so that the duration of treatment can be determined. In this way, the treatment regimen and dosing schedule can be rationally modified over the course of therapy so that the lowest amount of the drug exhibiting satisfactory effectiveness is administered, and so that administration is continued only for so long as is necessary to successfully treat the condition or disorder.

The present compositions can be used in combination therapies with opioids and other analgesics, including narcotic analgesics, Mu receptor antagonists, Kappa receptor antagonists, non-narcotic (i.e. non-addictive) analgesics, monamine uptake inhibitors, adenosine regulating agents, cannabinoid derivatives, Substance P antagonists, neurokinin-1 receptor antagonists and sodium channel blockers, among others. Preferred combination therapies comprise use of a composition of the invention with one or more compounds selected from aceclofenac, acemetacin, e-acetamidocaproic acid, acetaminophen, acetaminosalol, acetanilide, acetylsalicylic acid (aspirin), S-adenosylmethionine, alclofenac, alfentanil, allylprodine, alminoprofen, aloxiprin, alphaprodine, aluminum bis(acetylsalicylate), amfenac, aminochlorthenoxazin, 3-amino-4-hydroxybutyric acid, 2-amino-4-picoline, aminopropylon, aminopyrine, amixetrine, ammonium salicylate, ampiroxicam, amtolmetin guacil, anileridine, antipyrine, antipyrine salicylate, antrafenine, apazone, bendazac, benorylate, benoxaprofen, benzpiperylon, benzydamine, benzylmorphine, bermoprofen, bezitramide, α-bisabolol, bromfenac, p-bromoacetanilide, 5-bromosalicylic acid acetate, bromosaligenin, bucetin, bucloxic acid, bucolome, bufexamac, bumadizon, buprenorphine, butacetin, butibufen, butophanol, calcium acetylsalicylate, carbamazepine, carbiphene, carprofen, carsalam, chlorobutanol, chlorthenoxazin, choline salicylate, cinchophen, cinmetacin, ciramadol, clidanac, clometacin, clonitazene, clonixin, clopirac, clove, codeine, codeine methyl bromide, codeine phosphate, codeine sulfate, cropropamide, crotethamide, desomorphine, dexoxadrol, dextromoramide, dezocine, diampromide, diclofenac sodium, difenamizole, difenpiramide, diflunisal, dihydrocodeine, dihydrocodeinone enol acetate, dihydromorphine, dihydroxyaluminum acetylsalicylate, dimenoxadol, dimepheptanol, dimethylthiambutene, dioxaphetyl butyrate, dipipanone, diprocetyl, dipyrone, ditazol, droxicam, emorfazone, enfenamic acid, epirizole, eptazocine, etersalate, ethenzamide, ethoheptazine, ethoxazene, ethylmethylthiambutene, ethylmorphine, etodolac, etofenamate, etonitazene, eugenol, felbinac, fenbufen, fenclozic acid, fendosal, fenoprofen, fentanyl, fentiazac, fepradinol, feprazone, floctafenine, flufenamic acid, flunoxaprofen, fluoresone, flupirtine, fluproquazone, flurbiprofen, fosfosal, gentisic acid, glafenine, glucametacin, glycol salicylate, guaiazulene, hydrocodone, hydromorphone, hydroxypethidine, ibufenac, ibuprofen, ibuproxam, imidazole salicylate, indomethacin, indoprofen, isofezolac, isoladol, isomethadone, isonixin, isoxepac, isoxicam, ketobemidone, ketoprofen, ketorolac, p-lactophenetide, lefetamine, levorphanol, lofentanil, lonazolac, lornoxicam, loxoprofen, lysine acetylsalicylate, magnesium acetylsalicylate, meclofenamic acid, mefenamic acid, meperidine, meptazinol, mesalamine, metazocine, methadone hydrochloride, methotrimeprazine, metiazinic acid, metofoline, metopon, mofebutazone, mofezolac, morazone, morphine, morphine hydrochloride, morphine sulfate, morpholine salicylate, myrophine, nabumetone, nalbuphine, 1-naphthyl salicylate, naproxen, narceine, nefopam, nicomorphine, nifenazone, niflumic acid, nimesulide, 5′-nitro-2′-propoxyacetanilide, norlevorphanol, normethadone, normorphine, norpipanone, olsalazine, opium, oxaceprol, oxametacine, oxaprozin, oxycodone, oxymorphone, oxyphenbutazone, papaveretum, paranyline, parsalmide, pentazocine, perisoxal, phenacetin, phenadoxone, phenazocine, phenazopyridine hydrochloride, phenocoll, phenoperidine, phenopyrazone, phenyl acetylsalicylate, phenylbutazone, phenyl salicylate, phenyramidol, piketoprofen, piminodine, pipebuzone, piperylone, piprofen, pirazolac, piritramide, piroxicam, pranoprofen, proglumetacin, proheptazine, promedol, propacetamol, propiram, propoxyphene, propyphenazone, proquazone, protizinic acid, ramifenazone, remifentanil, rimazolium metilsulfate, salacetamide, salicin, salicylamide, salicylamide o-acetic acid, salicylsulfuric acid, salsalte, salverine, simetride, sodium salicylate, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiaramide, tilidine, tinoridine, tolfenamic acid, tolmetin, tramadol, tropesin, viminol, xenbucin, ximoprofen, zaltoprofen and zomepirac (see The Merck Index, 12th Edition (1996), Therapeutic Category and Biological Activity Index, lists therein headed “Analgesic”, “Anti-inflammatory” and “Antipyretic”).

Particularly preferred combination therapies comprise use of a composition of the invention, for example valdecoxib composition of the invention, with an opioid compound, more particularly where the opioid compound is codeine, meperidine, morphine or a derivative thereof.

The compound to be administered in combination with valdecoxib can be formulated separately from the valdecoxib or co-formulated with the valdecoxib in a composition of the invention. Where valdecoxib is co-formulated with a second drug, for example an opioid drug, the second drug can be formulated in immediate-release, rapid-onset, sustained-release or dual-release form.

In an embodiment of the invention, particularly where the cyclooxygenase-2 mediated condition is headache or migraine, the valdecoxib composition is administered in combination therapy with a vasomodulator, preferably a xanthine derivative having vasomodulatory effect, more preferably an alkylxanthine compound.

Combination therapies wherein an alkylxanthine compound is co-administered with a valdecoxib composition as provided herein are embraced by the present embodiment of the invention whether or not the alkylxanthine is a vasomodulator and whether or not the therapeutic effectiveness of the combination is to any degree attributable to a vasomodulatory effect. The term “alkylxanthine” herein embraces xanthine derivatives having one or more C₁₋₄ alkyl, preferably methyl, substituents, and pharmaceutically acceptable salts of such xanthine derivatives. Dimethylxanthines and trimethylxanthines, including caffeine, theobromine and theophylline, are especially preferred. Most preferably, the alkylxanthine compound is caffeine.

The total and relative dosage amounts of valdecoxib and of the vasomodulator or alkylxanthine are selected to be therapeutically and/or prophylactically effective for relief of pain associated with the headache or migraine. Suitable dosage amounts will depend on the severity of pain and the particular vasomodulator or alkylxanthine selected. For example, in a combination therapy with valdecoxib and caffeine, typically the valdecoxib will be administered in a daily dosage amount of about 1 mg to about 100 mg, preferably about 5 mg to about 50 mg, and the caffeine in a daily dosage amount of about 1 mg to about 500 mg, preferably about 10 mg to about 400 mg, more preferably about 20 mg to about 300 mg.

The vasomodulator or alkylxanthine component of the combination therapy can be administered in any suitable dosage form by any suitable route, preferably orally. The vasomodulator or alkylxanthine can optionally be coformulated with the valdecoxib in the composition of the invention. Thus a composition of the invention optionally comprises both valdecoxib and a vasomodulator or alkylxanthine such as caffeine, in total and relative amounts consistent with the dosage amounts set out hereinabove.

The phrase “in total and relative amounts effective to relieve pain”, with respect to amounts of valdecoxib and a vasomodulator or alkylxanthine in a composition of the present embodiment, means that these amounts are such that (a) together these components are effective to relieve pain, and (b) each component is or would be capable of contribution to a pain-relieving effect if the other component is or were not present in so great an amount as to obviate such contribution.

### EXAMPLES

The following examples illustrate aspects of the present invention but should not be construed as limitations.

### Example 1

Three valdecoxib composite granulations (G1-G3) were prepared according to the following procedure. Dry powder blends comprising valdecoxib and at least one of Avicel PH101, PVP (K29-32), and sodium lauryl sulfate (SLS) were prepared, and three granulation fluid batches were prepared, as shown in Table 1. The dry powder blends were wet granulated in a 2 liter Key granulator.

Valdecoxib composite granulation G1 was prepared with Eudragit® E PO, SLS and dibutyl sebecate dispersed in 97.6 g of water; this dispersion was added over four minutes to the dry powder blend with mixing to form a mixture. An additional 30 grams of water was then added to the mixture and the mixture was tray dried and hand passed through a 20 mesh screen to form valdecoxib composite granules.

Valdecoxib composite granulation G2 was prepared with PVP as a dry binder. Water was added to the dry powder blend over five minutes. Poor granulation uniformity was achieved with half of the material still dry and the other half over-granulated.

Valdecoxib composite granulation G3 was prepared with a granulation fluid comprising PVP dissolved in 60 grams of water. This solution was added to the dry powder blend over five minutes and an additional 30 grams of water was added over two minutes. This material was over-granulated with large agglomerates present.

TABLE 1
Valdecoxib Composite Granulations G1-G3

| | G1 | G2 | G3 |
|---|---|---|---|
| Dry powder | | | |
| Valdecoxib | 183.1 | 192.0 | 192.0 |
| Avicel PH101 | 98.6 | 93.0 | 93.0 |
| PVP, K29-32 | — | 15.0 | — |
| Sodium Lauryl | — | 3.0 | 3.0 |
| Sulfate | | | |
| Granulating Fluid | | | |
| Eudragit ® E PO | 20.0 | — | — |
| Sodium Lauryl | 1.4 | — | — |
| Sulfate | | | |
| Dibutyl Sebacate | 3.0 | — | — |
| Water | 127.6 | 73.2 | 90.0 |
| PVP, K29-32 | — | — | 15.0 |

### Example 2

Valdecoxib Fast-Melt Tablets (Batch A, hereinafter also referred to as Fast-Melt A), having components as shown in Table 2, were prepared according to the following procedure. Valdecoxib (457.75 g) and Avicel PH101 (226.92 g) were mixed together for two minutes in a Glatt granulator (main blade and chopper speeds set at 600 and 3000 rpm, respectively) to form a pre-mix. Eudragit® E PO (49 g), and citric acid (16.33 g) were added to a vessel containing 250 g of water to form a solution. The solution was added to the pre-mix (with continued mixing) at a substantially constant rate over a period of 8.5 minutes to form a wetted mixture. After addition of the solution was complete, the wetted mixture was further mixed for 1 minute to form a wet granulation. The resulting wet granulation was screened through an 18 mesh screen and dried in an oven or using a fluid bed dryer at 40° C. to form a dissolution-retarded valdecoxib composite. Valdecoxib composite (98.31 g) was then blended with 483.69 g of placebo granules (consisting of approximately 94% mannitol and 6% maltose) to form an intermediate blend; magnesium stearate, stearic acid, acesulfame potassium and peppermint flavor were added to the intermediate blend to form a tableting blend. Tablets were prepared by individually compressing 400 mg of the tableting blend to form tablets having an intermediate hardness of 1.5 kp. Resulting tablets were placed in a chamber maintained at 25° C. and 80% relative humidity for 1 hour, and at 40° C. and 30% relative humidity for a second hour.

TABLE 2
Composition (mg) of Fast-Melt A

| Component | Amount |
|---|---|
| Valdecoxib | 40 |
| Avicel PH101 | 19.83 |
| Eudragit ® E PO | 4.28 |
| Citric Acid | 1.43 |
| Mannitol | 302.46 |
| Maltose | 20 |
| Magnesium | 2 |
| stearate | |
| Stearic acid | 6 |
| Acesulfame | 2 |
| potassium | |
| Peppermint flavor | 2 |
| Total | 400 |

### Example 3

Valdecoxib Fast-Melt Tablets (Batch B, hereinafter also referred to as Fast-Melt B), having components as shown in Table 3, were prepared according to the following procedure. Valdecoxib (398.28 g) and Avicel PH101 (214.48 g) were mixed together for two minutes in a Glatt granulator (main blade and chopper speeds set at 600 and 3000 rpm, respectively) to form a pre-mix. Eudragit® E PO (112.15 g), sodium lauryl sulfate (7.88 g) and dibutyl sebecate (16.88 g) were added to a vessel containing 300 g of water to form a dispersion. The dispersion was added (with continued mixing) to the pre-mix at a substantially consistent rate over a period of 15 minutes to form a wetted mixture. After addition of the dispersion was complete, the wetted mixture was further mixed for 1 minute to form a wet granulation. The resulting wet granulation was screened through an 18 mesh screen and dried in an oven or using a fluid bed dryer at 40° C. to form a dissolution-retarded valdecoxib composite. The valdecoxib composite (112.99 g) was then blended with 469.01 g of placebo granules (approximately 94% mannitol and 6% maltose) to form an intermediate blend; magnesium stearate, stearic acid, acesulfame potassium and peppermint flavor were added to the intermediate blend to form a tableting blend. Tablets were then prepared by individually compressing 400 mg of the tableting blend to form tablets having an intermediate hardness of 1.5 kp. Resulting tablets were placed in a chamber maintained at 25° C. and 80% relative humidity for 1 hour, and at 40° C. and 30% relative humidity for a second hour.

TABLE 3
Composition (mg) of Fast-Melt B

| Component | Amount |
|---|---|
| Valdecoxib | 40 |
| Avicel PH101 | 21.54 |
| Eudragit ® E PO | 11.30 |
| Dibutyl sebacate | 1.70 |
| Sodim lauryl | 0.79 |
| sulfate | |
| Mannitol | 292.67 |
| Maltose | 20 |
| Magnesium | 2 |
| stearate | |
| Stearic acid | 6 |
| Acesulfame | 2 |
| potassium | |
| Peppermint flavor | 2 |
| Total | 400 |

### Example 4

Valdecoxib Fast-Melt Tablets (Batch C, hereinafter also referred to as Fast-Melt C), were prepared according to the following procedure. Valdecoxib and colloidal silicon dioxide were bag blended and passed through a Rotary Fines Granulator (Alexanderwerk Model RFG 150V) fitted with a 3.15 mm screen to form a first mixture. Sodium starch glycolate and sodium lauryl sulfate were bag blended to form a second mixture. The first and second mixtures were bag blended and passed through a Rotary Fines Granulator (Alexanderwerk Model RFG 150V) to form a third mixture. The third mixture was blended in a V-blender for 15 minutes and then roller compacted using an Alexanderwerk Roller Compactor (WP 120×40 V fitted with a 25 mm knurled roller, mass flow hopper) to form a granulation. Roller compactor process conditions were as follows: (a) hydraulic pressure: 60 bar; (b) feed screw: 56 RPM; (c) roller speed: 5 RPM; (d) granulator speed: 75 RPM. The resulting granulation was then classified using an 18 inch Sweeco Separator (fitted with US Standard 50 mesh sieve and 140 mesh sieve) and a 50/140 granule fraction was collected.

One thousand grams of the 50/140 granule fraction were fluid bed coated according to the following process. A dispersion was prepared having the following composition (% w/w): ethylcellulose (9.8); dibutyl sebecate (1.96); and absolute ethanol (to 100%). The 50/140 granule fraction was coated with 1133 g of dispersion using an Aeromatic Precision Coater, MP1 fluid bed unit to form coated granules having the composition shown in Table 4.

TABLE 4
Composition (%) of Coated Granules

| Component | Weight |
|---|---|
| Valdecoxib | 45 |
| Sodium starch | 41.4 |
| glycolate | |
| Sodium lauryl | 0.9 |
| sulfate | |
| Colloidal | 2.7 |
| silicon dioxide | |
| Ethylcellulose | 8.3 |
| Dibutyl | 1.7 |
| sebecate | |

Coated granules (89 mg) prepared as described above were blended with 299 mg of a placebo granulation (comprising approximately 93% mannitol and 7% maltose) and with magnesium stearate, stearic acid, acesulfame potassium and peppermint flavor to form a tableting blend. Fast-Melt C, having components as shown in Table 5, were prepared by individually compressing 400 mg of the tableting blend to an intermediate hardness of 1.5 kp. Resulting tablets were then placed in a chamber maintained at 25° C. and 80% relative humidity for 1 hour, and at 40° C. and 30% relative humidity for a second hour.

TABLE 5
Composition (mg) of Fast-Melt C

| Component | Amount |
|---|---|
| Valdecoxib | 40 |
| Sodium starch | 36.8 |
| glycolate | |
| Sodium lauryl sulfate | 0.8 |
| Colloidal silicon | 2.4 |
| dioxide | |
| Dibutyl sebecate | 1.6 |
| Ethylcellulose | 7.4 |
| Mannitol | 277.6 |
| Maltose | 21.4 |
| Magnesium stearate | 2 |
| Stearic acid | 6 |
| Acesulfame K | 2 |
| Peppermint flavor | 2 |

### Example 5

Valdecoxib Fast-Melt Tablets (Batch D, hereinafter referred to as Fast-Melt D), having components as shown in Table 6, were prepared according to the following procedure. Valdecoxib (900 g), colloidal silicon dioxide (50 g), and sodium starch glycolate (50 g) were admixed and dry milled to form a valdecoxib mixture. Sodium lauryl sulfate (5 g) and HPMC 2910 (50 g) were dissolved in a vessel containing water quantum sufficiat to form a solution; Eudragit® E PO (160 g), an additional 20 g of sodium lauryl sulfate and an additional 40 g of HPMC 2910 were then dispersed in the solution to form a dispersion. Additional water was added to result in a final Eudragit® E PO presence of about 15% (w/w) in the dispersion.

The valdecoxib mixture was then suspended in a fluid bed and the dispersion was top sprayed onto the mixture to form coated valdecoxib granules. The coated valdecoxib granules (112.99 g) were blended with 469.01 g of placebo granulation (approximately 93% mannitol and 7% maltose) to form an intermediate blend. Magnesium stearate, stearic acid, acesulfame K, and peppermint flavor were added to the intermediate blend to form a tableting blend. Tablets were then prepared by compressing 400 mg of the tableting blend to an intermediate hardness of 1.5 kp. Resulting tablets were then placed in a chamber maintained at 25° C. and 80% relative humidity for 1 hour, and at 40° C. and 30% relative humidity for a second hour.

TABLE 6
Composition (mg) of Fast-Melt D

| Component | Amount |
|---|---|
| Valdecoxib | 40 |
| Sodium starch | 2.22 |
| glycolate | |
| Sodium lauryl | 0.88 |
| sulfate | |
| Colloidal silicon | 0.22 |
| dioxide | |
| HPMC E5 | 2.22 |
| Eudragit ® E PO | 7.12 |
| Mannitol | 307.68 |
| Maltose | 23.66 |
| Magnesium stearate | 2 |
| Stearic acid | 6 |
| Acesulfame K | 2 |
| Peppermint flavor | 2 |

### Example 6

A comparative valdecoxib fast-melt tablet, Fast-Melt E, is prepared substantially as described in Example 2, however, no Eudragit® E PO is added to the solution/suspension. Eudragit® E PO is replaced in the final formulation by Avicel PH101.

### Example 7

A study was performed in order to determine pharmacokinetic properties of the Valdecoxib Fast-Melt A-D, in beagle dogs. Valdecoxib Fast-Melts A-D were individually administered to each of 4 dogs in a two-group partial cross-over study design. Venous blood was collected pre-dose, and at 0.5, 1, 1.5, 2, 2.5, 3, 4, 6, 8, 12 and 24 hours after oral dose administration. Plasma was separated from blood by centrifugation at 3000 G and samples were stored at −20° C. until analysis. Concentrations of valdecoxib in plasma were determined using an HPLC assay. Results are shown in Table 7.

TABLE 7
Pharmacokinetic properties of Valdecoxib
Fast-Melts A-D in Dogs

| Parameter | Fast-Melt A | Fast-Melt B | Fast-Melt C | Fast-Melt D |
|---|---|---|---|---|
| Cₘₐₓ (ng/ml) | 1410 | 2550 | 1100 | 2060 |
| AUC (h*ng/ml) | 4910 | 7540 | 3630 | 7160 |
| Tₘₐₓ (h) | 1.4 | 1.4 | 2.4 | 1.8 |

### Example 8

A study is performed in order to determine pharmacokinetic properties of Valdecoxib Fast-Melts A-D of Examples 2-5, by comparison with Valdecoxib Fast-Melt E of Example 6, in 24 healthy adult humans. Each subject is given one of the Fast-Melts, and venous blood is collected pre-dose, and at 0.5, 1, 1.5, 2, 2.5, 3, 4, 6, 8, 12, 16 and 24 hours after oral dose administration. Plasma is separated from blood by centrifugation at 3000 G and samples are stored at −20° C. until analysis. Concentrations of valdecoxib in plasma is determined using an HPLC assay. Analysis of blood from subjects taking Fast-Melts A-D results in a substantially similar Tₘₐₓ, substantially similar C_{max,} and substantially similar AUC, compared to analysis of blood from subjects taking Fast-Melt E.

### Example 9

Three valdecoxib composite granulations (G4-G6) were prepared according to the following procedure. Dry powder blends comprising valdecoxib, Avicel PH101, and a disintegrant (either crospovidone or croscarmellose sodium (Ac-Di-Sol)) were prepared along with three granulation fluid batches as shown in Table 8.

TABLE 8
Composition (g) of dry powder blends and granulating fluid
used to prepare valdecoxib composite granulations G4-G6

| | G4 | G5 | G6 |
|---|---|---|---|
| Dry Powder | | | |
| Valdecoxib | 398.28 | 368.56 | 368.56 |
| Avicel PH101 | 176.96 | 160.96 | 160.96 |
| Crospovidone | 37.5 | 37.5 | — |
| Croscarmellose | — | — | 37.5 |
| sodium | | | |
| Granulating Fluid | | | |
| Eudragit E PO | 112.5 | 150.0 | 150.0 |
| Sodium Lauryl | 7.88 | 10.49 | 10.49 |
| Sulfate | | | |
| Dibutyl Sebacate | 16.88 | 22.49 | 22.49 |
| Water | 300.0 | 400.0 | 400.0 |

The dry powder blends were then wet granulated with the granulation fluid as follows. Valdecoxib, Avicel PH101, and a disintegrant were added to a granulation bowl and premixed for two minutes at 600 RPM impeller speed and 3000 RPM chopper speed to form a dry mix. Granulation fluid was prepared by adding SLS and dibutyl sebacate to water with stirring; Eudragit E PO polymer was added slowly to the SLS solution. The granulation fluid was then sprayed into the dry powder at a spray rate of 30 ml/min, with addition times of 18.5 to 20 minutes, to form a wet granulation. The wet granulation was mixed, dried and subsequently de-lumped through a Quadro Comil.

Granulation particle sizes of valdecoxib composite granulations G4, G5 and G6 were assessed by sequentially sieving samples of granulations through screens of decreasing pore size. Data, indicating cumulative percentage, by weight, of granulation particles retained after passage through each sieve, are shown in Table 9.

TABLE 9
Amount (% weight) of granulation retained
in sieves of varying pore size

| Pore size | | | |
|---|---|---|---|
| (μm) | G4 | G5 | G6 |
| 850 | 0.30 | 0.89 | 0.30 |
| 425 | 8.36 | 23.49 | 11.00 |
| 250 | 24.58 | 54.61 | 36.90 |
| 180 | 46.47 | 77.11 | 64.30 |
| 106 | 81.29 | 96.33 | 92.30 |
| 75 | 90.35 | 99.31 | 97.60 |

Batches of the resulting valdecoxib composite granulations were then blended with a placebo granulation comprising approximately 93% mannitol and 7% maltose to form an intermediate blend. Magnesium stearate, stearic acid, acesulfame K, and peppermint flavor were added to the intermediate blend to form a tableting blend. Fast-Melt Tablets (Batches F-H; hereinafter also referred to as Fast Melts F, G and H, respectively) were then prepared by compressing an amount of tableting blend corresponding to between 39.9 and 40.1 mg of valdecoxib to an intermediate hardness of approximately 1.5 kp. Resulting tablets were placed in a chamber maintained at 25° C. and 80% relative humidity for one hour, and at 40° C. and 30% relative humidity for an additional one hour. Compositions of the Fast-Melts are shown in Table 10.

TABLE 10
Composition (mg) of Fast-Melts F-H

| Component | Fast-Melt F | Fast-Melt G | Fast-Melt H |
|---|---|---|---|
| Valdecoxib Composite | 75.2 | — | — |
| Granulation (G4) | | | |
| Valdecoxib Composite | — | 81.6 | — |
| Granulation (G5) | | | |
| Valdecoxib Composite | — | — | 81.6 |
| Granulation (G6) | | | |
| Mannitol | 290.8 | 284.8 | 284.8 |
| Maltose | 22 | 21.6 | 21.6 |
| Magnesium stearate | 2 | 2 | 2 |
| Stearic Acid | 6 | 6 | 6 |
| Acesulfame K | 2 | 2 | 2 |
| Peppermint flavor | 2 | 2 | 2 |
| Total | 400 | 400 | 400 |

### Example 10

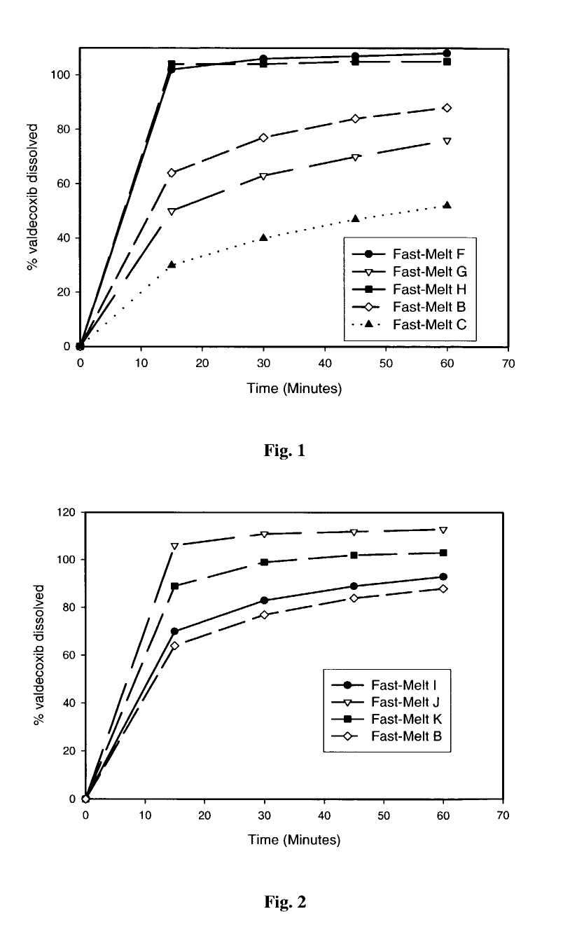

In vitro dissolution profiles of Fast-Melts F-H of Example 10 and Fast-Melts B and C of Examples 3 and 4, respectively, were determined using 1000 ml of 1% sodium lauryl sulfate solution and USP Type II Apparatus. Data are shown in FIG. 1. Overall, all fast-melt tablets tested exhibited rapid dissolution properties. Fast-Melts F and H exhibited most rapid dissolution with 100% of drug being dissolved after 15 minutes.

### Example 11

Three valdecoxib composite granulations (G7-G9) were prepared according to the following procedure. Dry powder blends comprising valdecoxib, Avicel PH101, and optionally a disintegrant (crospovidone) and three granulation fluid batches were prepared as shown in Table 11. The dry powder blends were then wet granulated with the granulation fluid as follows.

TABLE 11
Composition (g) of dry powder blends and granulating fluid
used to prepare valdecoxib composite granulations G7-G9

| | G7 | G8 | G9 |
|---|---|---|---|
| Dry Powder | | | |
| Valdecoxib | 364.16 | 412.71 | 408.77 |
| Avicel PH101 | 168.07 | 180.05 | 195.09 |
| Silicon Dioxide | 28.01 | 50.81 | 67.1 |
| Crospovidone | — | 33.87 | — |
| Granulating Fluid | | | |
| Eudragit E PO | 112.5 | 127.5 | 52.5 |
| Sodium Lauryl | 7.88 | 8.93 | 3.67 |
| Sulfate | | | |
| Dibutyl Sebacate | 16.88 | 19.13 | 7.87 |
| Water | 350.0 | 400.0 | 350 |
| Post Granulation | | | |
| Silicon Dioxide | 15 | 17 | 15 |
| Xylitol | 37.5 | — | — |

Valdecoxib, Avicel, and the optional disintegrants, sweetener and/or flavor were added to a granulation bowl and premixed for two minutes at 600 RPM impeller speed and 3000 RPM chopper speed to form a dry mix. Granulation fluid was prepared by adding SLS and dibutyl sebacate to water with stirring; Eudragit E PO polymer was added slowly and the granulation fluid was stirred for a period of about two hours. The granulation fluid was then sprayed into the dry powder with mixing to form a wet granulation; post granulation silicon dioxide and optionally xylitol were added. The wet granulation was dried and subsequently de-lumped to form valdecoxib composite granulations.

Batches of the resulting valdecoxib composite granulations were blended with a placebo granulation comprising approximately 93% mannitol and 7% maltose to form an intermediate blend. Magnesium stearate, stearic acid, acesulfame K, and peppermint flavor were added to the intermediate blend to form a tableting blend. Fast-Melt Tablets (Batches I-K; hereinafter also referred to as Fast Melts I, J and K, respectively) were prepared by compressing an amount of tableting blend corresponding to about 40 mg valdecoxib to an intermediate hardness of approximately 1.5 kp. Resulting tablets were placed in a chamber maintained at 25° C. and 80% relative humidity for one hour, and at 40° C. and 30% relative humidity for an additional one hour. Compositions of the Fast-Melts are shown in Table 12.

TABLE 12
Composition (mg) of Fast-Melts I-K

| Component | Fast-Melt I | Fast-Melt J | Fast-Melt K |
|---|---|---|---|
| Valdecoxib Composite | 82.4 | — | — |
| Granulation (G7) | | | |
| Valdecoxib Composite | — | 82.5 | — |
| Granulation (G8) | | | |
| Valdecoxib Composite | — | — | 73.1 |
| Granulation (G9) | | | |
| Mannitol | 284 | 284 | 292.4 |
| Maltose | 21.6 | 21.6 | 22 |
| Magnesium stearate | 2 | 2 | 2 |
| Stearic Acid | 6 | 6 | 6 |
| Acesulfame K | 2 | 2 | 2 |
| Peppermint flavor | 2 | 2 | 2 |
| Total | 400 | 400 | 400 |

### Example 12

In vitro dissolution profiles of Fast-Melts I-K of Example 11 and Fast-Melt B of Example 3 were determined using 1000 ml of 1% sodium lauryl sulfate solution and USP Type II Apparatus at 75 rpm. Data are shown in FIG. 2. Overall, all fast-melt tablets tested exhibited rapid dissolution properties. Fast-Melt Tablets J and K exhibited most rapid dissolution with more than 85% of drug being dissolved after 15 minutes.

### Example 13

Four valdecoxib composite granulations (G10-G13), as shown in Table 13, were prepared according to the following procedure. A dispersion was prepared by adding SLS and dibutyl sebacate to water with stirring. Eudragit EPO polymer was added slowly to the SLS solution. A portion of the Eudragit E PO was added initially, followed by one hour of mixing time; remaining Eudragit EPO was then added and the dispersion was allowed to mix for at least an additional two hours. Next, a solution was prepared by adding additional Eudragit E PO powder to water with mixing. Citric acid was added to the water and mixing was continued until a clear solution was obtained.

Valdecoxib, Avicel PH101 and, if used, silicon dioxide, sweetener and/or flavor, were added to a granulation bowl and pre-mixed for two minutes to form a dry powder mix. The dispersion prepared as described above was then sprayed into the powder, with mixing, over a period of approximately 11-13 minutes, to form wet granules. The wet granules were removed from the granulator bowl and were milled. A second granulation was performed on the wet granules using the Eudragit solution as granulating fluid. The Eudragit solution was sprayed on the granules over a period of several minutes. After addition, granules were mixed for one minute. The wet granules were then dried and subsequently de-lumped.

TABLE 13
Composition (g) of valdecoxib composite granulations G10-G13

| Composition | G10 | G11 | G12 | G13 |
|---|---|---|---|---|
| Valdecoxib | 422.9 | 355.9 | 355.9 | 355.9 |
| Avicel PH101 | 202.1 | 170.1 | 228.5 | 176.8 |
| Silicon Dioxide | 69.4 | 58.4 | — | 29.2 |
| Eudragit EPO | 127.5 | 107.3 | 107.3 | 107.3 |
| for suspension | | | | |
| Dibutyl | 19.1 | 16.1 | 16.1 | 16.1 |
| sebacate | | | | |
| Sodium Lauryl | 8.9 | 7.5 | 7.5 | 7.5 |
| sulfate | | | | |
| Eudragit EPO | 26.4 | 26.0 | 26.0 | 26.0 |
| for solution | | | | |
| Citric Acid | 8.8 | 8.7 | 8.7 | 8.7 |
| Acesulfame K | — | — | — | 7.5 |
| Peppermint | — | — | — | 15.0 |

Granule particle size present in valdecoxib composite granulations G1-G13 was assessed by sequentially sieving samples of the granulations through sieves of decreasing pore size. Data, indicating cumulative percentage, by weight, of granule particles retained after passage through each sieve, are shown in Table 14.

TABLE 14
Amount (% weight) of granulation retained
in sieves of varying pore size

| Pore size | | | | |
|---|---|---|---|---|
| (μm) | G10 | G11 | G12 | G13 |
| 850 | 0.3 | 0.2 | 0.0 | 0.1 |
| 425 | 11 | 24.8 | 27.8 | 19.4 |
| 250 | 36.9 | 46.2 | 59.9 | 38.9 |
| 180 | 64.3 | 61.5 | 81.5 | 58.5 |
| 106 | 92.3 | 80.2 | 99.1 | 87.7 |
| 75 | 97.6 | 85.6 | 99.9 | 96.1 |

An amount of a valdecoxib composite granulation was then blended with a dry granulation comprising approximately 93% mannitol and 7% maltose to form an intermediate blend. Magnesium stearate, stearic acid, acesulfame K, and peppermint flavor were added to the intermediate blend to form a tableting blend. Fast-Melt Tablets (Batches L-O; hereinafter also referred to Fast Melts L, M, N and O, respectively) were then prepared by compressing an amount of tableting blend corresponding to between 38.5 and 40 mg of valdecoxib to an intermediate hardness of approximately 1.5 kp. Resulting tablets were then placed in a chamber maintained at 25° C. and 80% relative humidity for one hour, and at 40° C. and 30% relative humidity for an additional one hour. Compositions of the tablets are shown in Table 15.

TABLE 15
Composition (mg) of Fast-Melts L-O

| | Fast-Melt | Fast-Melt | Fast-Melt | Fast-Melt |
|---|---|---|---|---|
| Component | L | M | N | O |
| Valdecoxib Composite | 83.6 | — | — | — |
| Granulation (G10) | | | | |
| Valdecoxib Composite | — | 81.2 | — | — |
| Granulation (G11) | | | | |
| Valdecoxib Composite | — | — | 81.2 | — |
| Granulation (G12) | | | | |
| Valdecoxib Composite | — | — | — | 81.2 |
| Granulation (G13) | | | | |
| Mannitol | 212.25 | 214 | 214 | 214 |
| Maltose | 16 | 16 | 16 | 16 |
| Magnesium stearate | 1.5 | 1.5 | 1.5 | 1.5 |
| Stearic Acid | 4.5 | 4.5 | 4.5 | 4.5 |
| Acesulfame K | 1.5 | 1.5 | 1.5 | 1.5 |
| Peppermint flavor | 1.5 | 1.5 | 1.5 | 1.5 |
| Total | 400 | 400 | 400 | 400 |

### Example 14

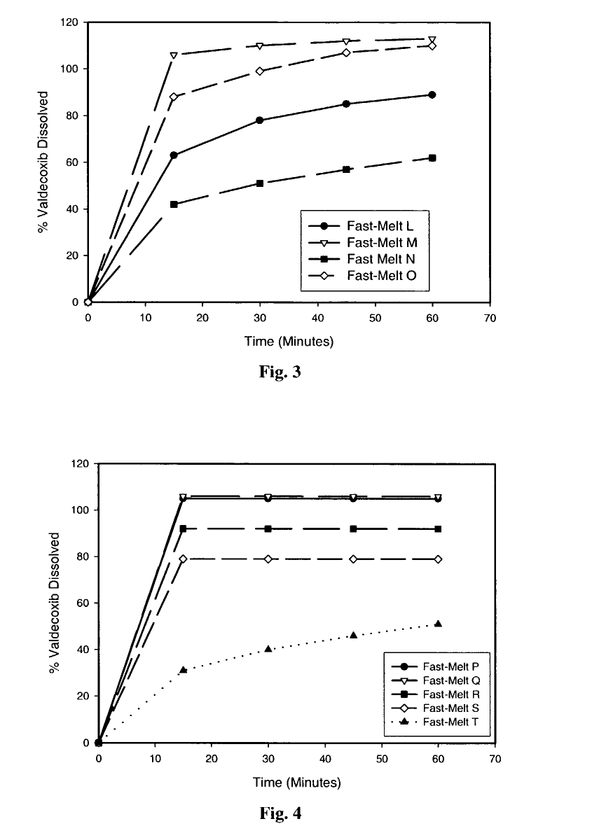

In vitro dissolution profiles of Fast-Melts L-O of Example 13 were determined using 1000 ml of 1% sodium lauryl sulfate solution and USP Type II Apparatus. Data are shown in FIG. 3. Fast-Melt Tablets M and O exhibited the fasted dissolution times out of the four tablet formulations.

### Example 15

Five valdecoxib composite granulations (G14-G18), as shown in Table 16, were prepared according to the following procedure. Valdecoxib, Avicel and, if used, disintegrants, sweetener and/or flavor, were added to a granulation bowl and premixed for two minutes to form a dry granulation mixture. A dispersion was prepared by adding SLS and dibutyl sebacate to a vessel of water with stirring. Eudragit EPO polymer was added slowly to the SLS dispersion with mixing. The dispersion was then sprayed onto the granulation mixture at a spray rate of 30 ml/min over a period of approximately 20 minutes to form a wet granulation. The wet granulation was mixed, dried and subsequently de-lumped to form valdecoxib composite granulations.

TABLE 16
Composition (g) of valdecoxib composite granulations G14-G18

| Composition | G14 | G15 | G16 | G17 | G18 |
|---|---|---|---|---|---|
| Valdecoxib | 368.6 | 368.6 | 368.6 | 368.6 | 368.6 |
| Avicel PH101 | 146 | 138.4 | 177.5 | 155 | 198.5 |
| Eudragit EPO | 150 | 150 | 150 | 150 | 150 |
| Croscarmellose | 37.5 | 37.5 | 21 | 21 | — |
| sodium | | | | | |
| Dibutyl | 22.5 | 22.5 | 22.5 | 22.5 | 22.5 |
| sebacate | | | | | |
| Sodium Lauryl | 10.5 | 10.5 | 10.5 | 10.5 | 10.5 |
| sulfate | | | | | |
| Acesulfame K | — | 7.5 | — | 7.5 | — |
| Peppermint | — | 15 | — | 15 | — |

Particle size of granules present in valdecoxib composite granulations G14-G18 was assessed by sequentially sieving samples of the granulations through sieves of decreasing pore size. Data, indicating cumulative percentage, by weight, of granulation retained after passage through each sieve, are shown in Table 17.

TABLE 17
Amount (% weight) of granulation retained
in sieves of varying pore size

| Pore size | | | | | |
|---|---|---|---|---|---|
| (μm) | G14 | G15 | G16 | G17 | G18 |
| 850 | 0.1 | 0.3 | 0.5 | 0.1 | 0.2 |
| 425 | 2.3 | 7.3 | 5.7 | 27.2 | 16.1 |
| 250 | 9.0 | 34.5 | 29.3 | 78.9 | 62.4 |
| 180 | 62.1 | 83.0 | 77.8 | 94.4 | 90.1 |
| 106 | 91.4 | 98.4 | 96.4 | 99.7 | 99.6 |
| 75 | 97.9 | 99.5 | 99.1 | 100 | 100 |

An amount of a valdecoxib composite granulation was blended with a placebo granulation (comprising approximately 93% mannitol and 7% maltose) to form an intermediate blend. Magnesium stearate, stearic acid, acesulfame K, and peppermint flavor were added to the intermediate blend to form a tableting blend. Fast-Melt Tablets (Batches P-T) were then prepared by compressing an amount of the tableting blend corresponding to about 40 mg of valdecoxib to an intermediate hardness of approximately 1.5 kp. Resulting tablets were then placed in a chamber maintained at 25° C. and 80% relative hunidity for one hour, and at 40° C. and 30% relative humidity for an additional one hour. Compositions of the tablets are shown in Table 18.

TABLE 18
Composition (mg) of Fast-Melts P-T

| | | | | | |
|---|---|---|---|---|---|
| | Fast- | | | | |
| | Melt | Fast-Melt | Fast-Melt | Fast-Melt | Fast-Melt |
| Component | P | Q | R | S | T |

Valdecoxib Composite Granulation

| | | | | | |
|---|---|---|---|---|---|
| G14 | 81.2 | — | — | — | — |
| G15 | — | 81.3 | — | — | — |
| G16 | — | — | 81.2 | — | — |
| G17 | — | — | — | 81.2 | — |
| G18 | — | — | — | — | 81.6 |
| Mannitol | 284.8 | 284.8 | 284.8 | 284.8 | 284.8 |
| Maltose | 21.6 | 21.6 | 21.6 | 21.6 | 21.6 |
| Magnesium | 2 | 2 | 2 | 2 | 2 |
| stearate | | | | | |
| Stearic Acid | 6 | 6 | 6 | 6 | 6 |
| Acesulfame K | 2 | 2 | 2 | 2 | 2 |
| Peppermint | 2 | 2 | 2 | 2 | 2 |
| flavor | | | | | |
| Total | 400 | 400 | 400 | 400 | 400 |

### Example 16

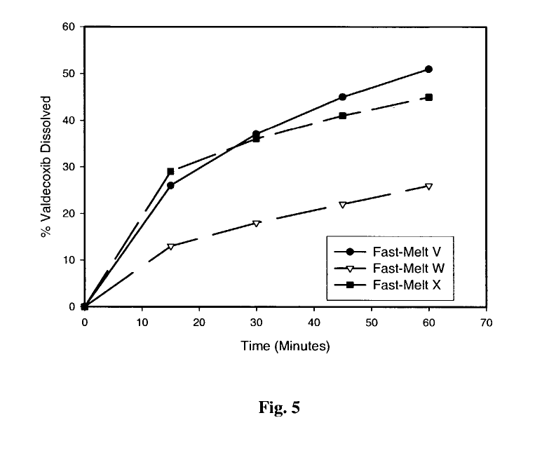

In vitro dissolution profiles of Fast-Melts P-T of Example 15 were determined using 1000 ml of 1% sodium lauryl sulfate solution and USP Type II Apparatus. Data are shown in FIG. 4. Fast-Melt Tablets comprising croscarmellose sodium exhibited very rapid valdecoxib dissolution.

### Example 17

Valdecoxib Fast-Melt Tablets (Batch U, hereinafter also referred to as Fast-Melt U), having components as shown in Table 19, were prepared according to the following procedure. Valdecoxib (368.56) and Avicel PH101 (198.46 g) were mixed together in a Glatt granulator to form a pre-mix. Eudragit® E PO (150 g), sodium lauryl sulfate (10.49 g) and dibutyl sebecate (22.49 g) were added to a vessel containing of water to form a suspension. The suspension was added (with continued mixing) to the pre-mix at a substantially consistent rate over a period of 15 minutes to form a wetted mixture. After addition of the suspension was complete, the wetted mixture was further mixed for 1 minute to form a wet granulation. The resulting wet granulation was screened through an 18 mesh screen and dried in an oven or using a fluid bed dryer at 40° C. to form a dissolution-retarded valdecoxib composite. The valdecoxib composite (122.10 g) was then blended with 459.90 g of placebo granules (approximately 94% mannitol and 6% maltose) to form an intermediate blend; magnesium stearate, stearic acid, acesulfame potassium and peppermint flavor were added to the intermediate blend to form a tableting blend. Tablets were then prepared by individually compressing an amount of the tableting blend corresponding to 40 mg of valdecoxib to form tablets having an intermediate hardness of 1.5 kp. Resulting tablets were placed in a chamber maintained at 25° C. and 80% relative humidity for 1 hour, and at 40° C. and 30% relative humidity for a second hour.

TABLE 19
Composition (mg) of Fast-Melt U

| Component | Amount |
|---|---|
| Valdecoxib | 40 |
| Avicel PH101 | 21.6 |
| Eudragit ® E PO | 16.4 |
| Dibutyl sebacate | 2.4 |
| Sodim lauryl | 1.2 |
| sulfate | |
| Mannitol | 285 |
| Maltose | 21.4 |
| Magnesium | 2 |
| stearate | |
| Stearic acid | 6 |
| Acesulfame | 2 |
| potassium | |
| Peppermint flavor | 2 |
| Total | 400 |

### Example 18

Three valdecoxib composite granulations, G19-G21, as shown in Table 20, were prepared according to the following procedure. Valdecoxib, Avicel, and if used, a disintegrant, were added to a granulation bowl and premixed for two minutes to form a dry granulation mixture. A dispersion was prepared by placing mannitol and Surelease®, an ethylcellulose dispersion, in a vessel with stirring. The dispersion was then added to the granulation mixture over a period of about 13.5 minutes, with mixing, to form a wet granulation. The wet granulation was then dried and de-lumped to form valdecoxib composite granulations.

TABLE 20
Composition (g) of valdecoxib composite granulations G19-G21

| Composition | G19 | G20 | G21 |
|---|---|---|---|
| Valdecoxib | 426.56 | 419.25 | 419.2 |
| Avicel PH101 | 229.69 | 225.75 | 188.25 |
| Surelease ® | 330 | 330 | 330 |
| Crospovidione | — | — | 37.5 |
| Mannitol | 11.25 | 22.5 | 22.5 |

Granule particle size present in valdecoxib composite granulations G19-G21 was asssessed by sequentially sieving samples of granulations through seives of decreasing pore size. Data, indicating cumulative percentage, by weight, of granulation retained after passage through each sieve, are shown in Table 21.

TABLE 21
Amount (% weight) of granulation retained
in sieves of varying pore size

| Pore size | | | |
|---|---|---|---|
| (μm) | G19 | G20 | G21 |
| 850 | 0.1 | 0.3 | 0.5 |
| 425 | 5.4 | 16.4 | 23.3 |
| 250 | 16.3 | 39.7 | 51.7 |
| 180 | 44.3 | 69.4 | 72.7 |
| 106 | 68.8 | 93.1 | 84.8 |
| 75 | 80.7 | 97.9 | 87.8 |

Valdecoxib composite granulation (52.75 g) was blended with 238.25 g of placebo granulation (comprising approximately 93% mannitol and 7% maltose) to form an intermediate blend. Magnesium stearate, stearic acid, acesulfame K, and peppermint flavor were added to the intermediate blend to form a tableting blend. Fast-Melt Tablets (Batches V-X) were then prepared by compressing an amount of the tableting blend corresponding to 40 mg of valdecoxib to an intermediate hardness of approximately 1.5 kp Resulting tablets were then placed in a chamber maintained at 25° C. and 80% relative humidity for one hour, and at 40° C. and 30% relative humidity for an additional one hour.

### Example 19

Fast-Melts V-X of Example 18 were evaluated in an in vitro dissolution assay as described in Example 16. Data are shown in FIG. 5. All Fast-Melts released less than 30% of initial valdecoxib present after 15 minutes in the dissolution assay.

### Example 20

Fast-Melts H, J, L and U of Examples 9, 11, 13, and 17, respectively, were administered to dogs and oral bioavailability parameters were determined. Bioavailability parameters were also determined for a commercially available 40 mg Bextra® tablet. Data, shown in Table 22, are reported as a percentage relative to the corresponding data for the Bextra® tablet. Importantly, because of differences in gastrointestinal systems between dogs and humans, these data are not likely representative of relative bioavailability as would be observed in humans.

TABLE 22
Relative bioavailability (%) of Fast-Melts H, J, L and U

| | Fast-Melt | Fast-Melt | Fast-Melt | Fast-Melt |
|---|---|---|---|---|
| | H | J | L | U |
| Relative AUC | 56.5 | 69.8 | 58.7 | 62.0 |
| Relative Cₘₐₓ | 64.4 | 71.0 | 56.9 | 67.5 |

### Example 21

Fast-Melts H, J, L and U of Examples 9, 11, 13, and 17, respectively, were evaluated in an organoleptic evaluation study according to the following procedure. Four to five professional sensory panelists were selected and each panelist was given a Fast-Melt tablet to place on his/her tongue. The panelist gently rolled the tablet against the roof of his/her mouth without chewing, and simultaneously recorded sensory information and time to complete disintegration. Sensory information included organoleptic attributes associated with each tablet such as flavor quality, bitterness, fullness, texture, mouth feel and aftertaste. Each of these attributes were defined along a categorical unit scale of 1-5 to express perceptual differences from other commercially marketed melt products, by comparison with valdecoxib fast-melt tablets which comprised one of cherry, strawberry, orange, peppermint, or spearmint, but which comprised no dissolution retardant (comparator taste-masked tablets), and by comparison with other fast-melt tablets not relevant to the present invention.

After total disintegration of a tablet, the panelist recorded sensory aftertaste over a period of 30 minutes. Each fast melt was evaluated in triplicate and all samples were coded for presentation to panelists.

Average disintegration times for each of Fast-Melts H, J, L and U are shown in Table 23.

TABLE 23
Disintegration times for Fast-Melts H, J, L and U

| | Fast-Melt | Fast-Melt | Fast-Melt | Fast-Melt |
|---|---|---|---|---|
| | H | J | L | U |
| Disintegration time | 23.6 | 18.8 | 21.7 | 19.4 |
| (seconds) | | | | |

Overall, valdecoxib Fast-Melts H, J, L and U exhibited higher flavor quality than did any of the comparator taste-masked valdecoxib tablets comprising a flavor agent but no dissolution retardant (data not shown).

### Example 22

Fast-Melt H of Example 9 was individually administered to 23 human subjects. Oral bioavailability parameters were determined and compared with those of a 40 mg commercial Bextra® tablet. Data are shown in Table 24.

TABLE 24
Oral bioavailability of Fast-Melt H and a
40 mg Bextra ® tablet in humans

| Parameter | Fast-Melt H | Bextra ® tablet |
|---|---|---|
| Tₘₐₓ (hr) | 4.5 | 3.3 |
| Cₘₐₓ (ng/ml) | 421 | 468 |
| AUC | 6171 | 6126 |
| (ng/ml)/hr | | |

These data indicate that Fast-Melt H and the commercial Bextra® tablet are similarly bioavailable upon oral administration to a human subject.

What is claimed is:

1. An oral fast-melt composition comprising
(a) particulate valdecoxib in a therapeutically effective amount,
(b) at least one pharmaceutically acceptable dissolution retardant, and
(c) at least one pharmaceutically acceptable excipient which exhibits rapid oral dissolution;
wherein the composition is organoleptically acceptable.

2. The composition of claim 1 which, when placed in United States Pharmacopeia 24 in vitro disintegration Test Number 701, exhibits a disintegration time of less than about 300 seconds.

3. The composition of claim 1 which, when placed in United States Pharmacopeia 24 in vitro disintegration Test Number 701, exhibits a disintegration time of less than about 200 seconds.

4. The composition of claim 1 which, when placed in United States Pharmacopeia 24 in vitro disintegration Test Number 701, exhibits a disintegration time of less than about 100 seconds.

5. The composition of claim 1 which disintegrates within about 60 seconds after placement in the oral cavity of a human subject.

6. The composition of claim 1 which disintegrates within about 30 seconds after placement in the oral cavity of a human subject.

7. The composition of claim 1 which disintegrates within about 15 seconds after placement in the oral cavity of a human subject.

8. The composition of claim 1 which upon oral administration to a human subject results in a valdecoxib concentration attaining a threshold for therapeutic effect within about 0.5 h of administration.

9. The composition of claim 1 which upon oral administration to a human subject results in a valdecoxib concentration attaining a threshold for therapeutic effect within about 0.3 h of administration.

10. The composition of claim 1 which upon oral administration to a human subject results in a time to reach maximum blood serum concentration (Tₘₐₓ) not greater than about 5 h.

11. The composition of claim 1 which upon oral administration to a human subject results in a time to reach maximum blood serum concentration (Tₘₐₓ) not greater than about 4.5 h.

12. The composition of claim 1 which upon oral administration to a human subject results in a maximum blood serum concentration (Cₘₐₓ) not less than about 100 ng/ml.

13. The composition of claim 1 which upon oral administration to a human subject results in a maximum blood serum concentration (Cₘₐₓ) not less than about 200 ng/ml.

14. The composition of claim 1 which upon oral administration to a human subject results in a maximum blood serum concentration (Cₘₐₓ) not less than about 300 ng/ml.

15. The composition of claim 1 wherein the at least one pharmaceutically acceptable dissolution retardant is a polymer.

16. The composition of claim 15 wherein the polymer is present in a total amount of about 0.5% to about 15%, by weight.

17. The composition of claim 15 wherein the polymer is present in a total amount of about 0.75% to about 10%, by weight.

18. The composition of claim 15 wherein the polymer is present in a total amount of about 1.0% to about 5%, by weight.

19. The composition of claim 1 wherein the at least one pharmaceutically acceptable dissolution retardant is selected from the group consisting of ethylcellulose, hydroxypropylmethylcellulose, polyvinylpyrrolidone, Eudragit® EP O and equivalent polymethacrylate products, hydroxypropylethylcellulose and hydroxypropylcellulose.

20. The composition of claim 1 wherein the at least one pharmaceutically acceptable dissolution retardant is Eudragit® EP O or an equivalent polymethacrylate product.

21. The composition of claim 1 wherein the at least one pharmaceutically acceptable excipient which exhibits rapid oral dissolution is a carbohydrate.

22. The composition of claim 1 wherein the at least one pharmaceutically acceptable excipient which exhibits rapid oral dissolution is a saccharide.

23. The composition of claim 1 wherein the at least one pharmaceutically acceptable excipient which exhibits rapid oral dissolution is selected from the group consisting of maltose, maltitol, sorbitol, lactose and mannitol.

24. The composition of claim 1 wherein the at least one pharmaceutically acceptable excipient which exhibits rapid oral dissolution comprises a saccharide of high moldability and a saccharide of low moldability.

25. The composition of claim 24 wherein the weight ratio of saccharide of high moldability to saccharide of low moldability is about 2 to about 20 parts of saccharide of high moldability per 100 parts of saccharide of low moldability.

26. The composition of claim 24 wherein the weight ratio of saccharide of high moldability to saccharide of low moldability is about 5 to about 10 parts of saccharide of high moldability per 100 parts of saccharide of low moldability.

27. The composition of claim 24 wherein the weight ratio of saccharide of high moldability to saccharide of low moldability is about 5 to about 7.5 parts of saccharide of high moldability per 100 parts of saccharide of low moldability.

28. The composition of claim 1 wherein the at least one pharmaceutically acceptable excipient which exhibits rapid oral dissolution is present in a total amount of about 10% to about 90%, by weight.

29. The composition of claim 1 wherein the at least one pharmaceutically acceptable excipient which exhibits rapid oral dissolution is present in a total amount of about 10% to about 80%, by weight.

30. The composition of claim 1 wherein the at least one pharmaceutically acceptable excipient which exhibits rapid oral dissolution is present in a total amount of about 10% to about 75%, by weight.

31. The composition of claim 1 having a hardness of about 1 to about 10 kp.

32. The composition of claim 1 having a hardness of about 1 to about 6 kp.

33. The composition of claim 1 wherein the valdecoxib is present in an amount of about 5 to about 50 mg.

34. The composition of claim 1 wherein the valdecoxib has a D₉₀ particle size not greater than about 75 μm.

35. The composition of claim 1 wherein the valdecoxib has a D₉₀ particle of about 1 to about 10 μm.

36. A process for preparing an intraorally disintegrating valdecoxib fast-melt composition, the process comprising:
a step of providing valdecoxib in particulate form;
a step of adding to the valdecoxib at least one pharmaceutically acceptable dissolution retardant to form a valdecoxib composite;
a step of admixing with the valdecoxib composite at least one pharmaceutically acceptable excipient that exhibits rapid oral dissolution, said admixing step forming a tableting blend;
a step of granulating the valdecoxib, valdecoxib composite, or tableting blend; and
a step of compressing the tableting blend to form the fast-melt composition;
wherein said granulating step occurs prior to, simultaneously with, and/or after said step of adding the dissolution retardant.

37. The process of claim 36 wherein the granulating step comprises wet granulation.

38. The process of claim 37 further comprising a step of drying the valdecoxib composite or tableting blend during and/or after the wet granulation step.

39. The process of claim 38 wherein the drying step comprises tray drying in an oven.

40. The process of claim 38 wherein the drying step comprises fluid bed drying.

41. The process of claim 37 wherein the wet granulation step comprises high shear wet granulation.

42. The process of claim 37 wherein the wet granulation step comprises fluid bed granulation.

43. The process of claim 36 wherein the granulation step comprises dry granulation.

44. The process of claim 43 wherein the dry granulation step comprises roller compaction.

45. The process of claim 44 wherein the dry granulation step comprises slugging.

46. The process of claim 36 wherein the at least one pharmaceutically acceptable dissolution retardant is a polymer.

47. The process of claim 36 wherein the at least one pharmaceutically acceptable dissolution retardant is selected from the group consisting of ethylcellulose, hydroxypropylmethylcellulose, polyvinylpyrrolidone, Eudragit® EP O and equivalent polymethacrylate products, hydroxypropylethylcellulose and hydroxypropylcellulose.

48. The process of claim 36 wherein the at least one dissolution retardant is Eudragit® EP O or an equivalent polymethacrylate product.

49. The process of claim 36 wherein the dissolution retardant is added in a total amount of about 0.5% to about 15%, by weight of the composition.

50. The process of claim 36 wherein the dissolution retardant is added in a total amount of about 0.75% to about 10%, by weight of the composition.

51. The process of claim 36 wherein the dissolution retardant is added in a total amount of about 1.0% to about 5%, by weight of the composition.

52. The process of claim 36 wherein the at least one pharmaceutically acceptable excipient which exhibits rapid oral dissolution is a carbohydrate.

53. The process of claim 36 wherein the at least one pharmaceutically acceptable excipient which exhibits rapid oral dissolution is a saccharide.

54. The process of claim 36 wherein the at least one pharmaceutically acceptable excipient which exhibits rapid oral dissolution is selected from the group consisting of maltose, maltitol, sorbitol, lactose and mannitol.

55. The process of claim 36 wherein the at least one pharmaceutically acceptable excipient which exhibits rapid oral dissolution comprises a saccharide of high moldability and a saccharide of low moldability.

56. The process of claim 55 wherein the weight ratio of saccharide of high moldability to saccharide of low moldability is about 2 to about 20 parts of saccharide of high moldability per 100 parts of saccharide of low moldability.

57. The process of claim 36 wherein the weight ratio of saccharide of high moldability to saccharide of low moldability is about 5 to about 10 parts of saccharide of high moldability per 100 parts of saccharide of low moldability.

58. The process of claim 36 wherein the weight ratio of saccharide of high moldability to saccharide of low moldability is about 5 to about 7.5 parts of saccharide of high moldability per 100 parts of saccharide of low moldability.

59. The process of claim 36 wherein the at least one pharmaceutically acceptable excipient which exhibits rapid oral dissolution is admixed in a total amount of about 10% to about 90%, by weight of the composition.

60. The process of claim 36 wherein the at least one pharmaceutically acceptable excipient which exhibits rapid oral dissolution is admixed in a total amount of about 10% to about 80%, by weight of the composition.

61. The process of claim 36 wherein the at least one pharmaceutically acceptable excipient which exhibits rapid oral dissolution is admixed in a total amount of about 10% to about 75%, by weight of the composition.

62. A valdecoxib fast-melt composition prepared according to the process of claim 36.

63. A method for treating or preventing a medical condition or disorder in a subject where treatment with a cyclooxygenase-2 inhibitory drug is indicated, comprising oral administration to the subject a fast-melt of claim 1.

64. A method for treating or preventing a medical condition or disorder in a subject where treatment with a cyclooxygenase-2 inhibitory drug is indicated, comprising oral administration to the subject a composition of claim 62.
